(19) United States
(12) Patent Application Publication
(10) Pub. No.: US 2023/0389997 A1
(43) Pub. Date: Dec. 7, 2023

(54) POSITION TRACKING SENSOR HAVING TAPPED ANGLED COIL

(71) Applicants: Sean Morgan, Golden Valley, MN (US); Wai Welly Chou, Minneapolis, MN (US); Roshan Shrestha, Minneapolis, MN (US); Peter Mueller, Minneapolis, MN (US); Richard John Barthel, Minneapolis, MN (US); Syewon Sei Weah, Minneapolis, MN (US); Lev Koyrakh, Plymouth, MN (US); Andrew Gadbois, Hugo, MN (US)

(72) Inventors: Sean Morgan, Golden Valley, MN (US); Wai Welly Chou, Minneapolis, MN (US); Roshan Shrestha, Minneapolis, MN (US); Peter Mueller, Minneapolis, MN (US); Richard John Barthel, Minneapolis, MN (US); Syewon Sei Weah, Minneapolis, MN (US); Lev Koyrakh, Plymouth, MN (US); Andrew Gadbois, Hugo, MN (US)

(21) Appl. No.: 18/330,860

(22) Filed: Jun. 7, 2023

Related U.S. Application Data
(60) Provisional application No. 63/349,743, filed on Jun. 7, 2022.

(51) Int. Cl. A61B 34/20; A61B 5/06

(52) U.S. Cl. CPC A61B 34/20; A61B 5/062; A61B 2034/2051; A61B 2034/2072

(57) ABSTRACT

A sensor includes a core oriented along a first vector corresponding to an axis of the core. A slanted coil has windings around the core at an angle to the axis of the core along a second vector oriented at a substantial angle to the direction of the windings. At least one middle conductor is coupled to the windings to enable dividing the windings into sub-coils about the middle conductors. The sub-coils may have different slants with respect to the direction of the windings. A pair of end conductors are coupled to opposite ends of the coils such that signals obtained from the middle and end conductors provide sufficient information to determine roll about the axis of the core.

### RELATED APPLICATION

This application claims priority to U.S. Provisional Application Ser. No. 63/349,743 (entitled Position Tracking Sensor Having Tapped Angled Coil, filed Jun. 7, 2022) which is incorporated herein by reference.

### BACKGROUND

Electromagnetic tracking systems are widely used with tracking sensors placed within surgical tools or on and around the patient during medical procedures. Many electromagnetic field sensors used for tracking are made with wire coils wrapped around various cores or the surgical tools themselves. The latter are typically called air core or hollow core sensors. Sensors can include several coil sub-assemblies. Single coil sensors usually cannot resolve a roll around the coils axis degree of freedom (DOF) and are called 5DOF, where three degrees of freedom encode the spatial position of the sensor and two degrees of freedom encode the orientation of the coil axis (for example pitch and yaw). To determine the roll around the axis (also called just “roll”), additional coil sub-assemblies are typically used within the sensor assembly. These coils are usually placed close together at some angles to each other or at a distance from one another in a rigid assembly in order to enable the determination of the roll.

### SUMMARY

A sensor includes a core oriented along a first vector corresponding to an axis of the core. A slanted coil has windings around the core at an angle to the axis of the core along a second vector oriented at a substantial angle to the direction of the windings. At least one middle conductor is coupled to the windings to enable dividing the windings into sub-coils about the middle conductors. The sub-coils may have different slants with respect to the direction of the windings. A pair of end conductors are coupled to opposite ends of the coils such that signals obtained from the middle and end conductors provide sufficient information to determine roll about the axis of the core.

### BRIEF DESCRIPTION OF THE DRAWINGS

FIG. 1 is a diagram of an electromagnetic tracking system in the clinical context according to an example embodiment.

FIG. 2 is a PRIOR ART diagram of a sensor having a straight wound coil.

FIG. 3 is a diagram of a sensor having a coil wound at an angle to a core axis according to an example embodiment.

FIG. 4 is a diagram of a sensor having a coil winding orientation V1 determined as a vector connecting the centers of two sub-coils according to an example embodiment,

FIG. 5 is a diagram of a sensor having two sub-coils wound at different orientations with respect to the core according to an example embodiment.

FIG. 6 is a block schematic diagram of a computer system to drive antennas, collect sensor information, and derive position and orientation information and for performing methods and algorithms according to example embodiments

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

An improved position sensor is capable of providing signals from which localization can be determined with six degrees of freedom (DOE). A single axis sensor, which can be a single coil, has conductive windings wrapped at an angle to its core axis with one or more conductors attached between the two end conductors forming one or more sub-coils. The coil wrap angle can be somewhere between 3 and 87 degrees to the direction of the coil axis to allow a reliable determination of the roll degree of freedom of the sensor. Such a slanted coil with one or more conductors connected at or near the center can be used to enable 6DOF localization. The slanted coil's orientation is now determined by a slant angle relative to the coil axis, which no longer coincides with the coil axis. The coil axis direction can be determined as a vector connecting the two or more separate sub-coils: one that is formed by the conductors connected to one end of the coil and to the middle conductor, and another formed by the conductors connected to other end of the coil and to the middle for the case of a single middle conductor.

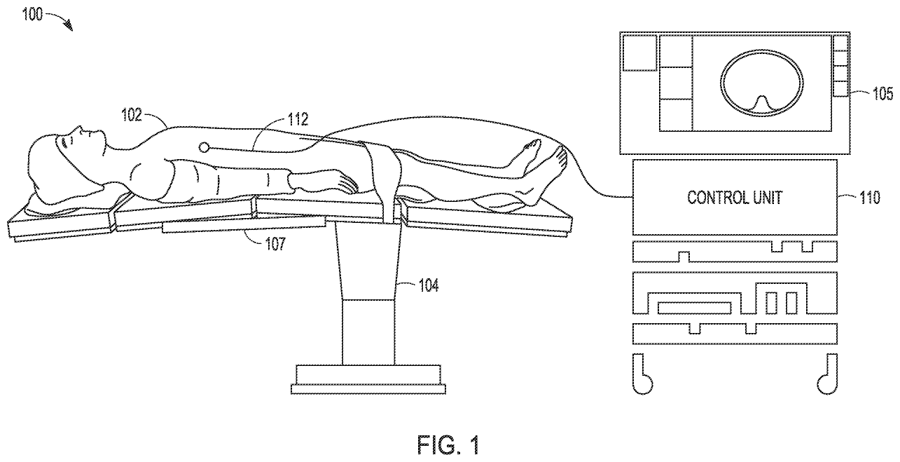

FIG. 1 is a diagram of an electromagnetic (EM) tracking system 100 in a clinical context according to an example embodiment showing a patient 102 positioned on a stand 104. A field generating antenna 107 is placed under the patient 102. The field generating antenna 107 may include multiple coils that may be used to generate magnetic fields such as described in U.S. patent application Ser. No. 17/278,466 (Published Application US2022-0037085) and U.S. patent application Ser. No. 18/177,215 which are incorporated herein by reference for their teaching of field generating antennas.

A surgical tool 112 inside the patient's body is shown on a procedure application screen 105 overlayed on an anatomical map of the patient, allowing GPS-like navigation of the tool 112 within the patient 102.

Coil sensors may be embedded in various tools, such as a catheters, surgical needles, guidewires, etc., or placed on tools outside the patient or attached to the patient, allowing tool tracking in real time h measuring voltage generated in the coils in response to being subjected to magnetic fields. Coils sensors are tracked by the electromagnetic tracking system 100, Which consists of the field generator (FG) or antenna 107 which creates a plurality of electromagnetic fields, and a control unit 110 which drives currents through the field generator 107, receives sensed inputs from the coil sensors (not visible in FIG. 1) in the tool 112, and processes the signals in order to determine position and orientation (P&O) of the sensor. The P&O information can be determined for each sensor depending on the sensor construction and the number of the electromagnetic fields created by the FG 107. Determination of P&O information for a single coil sensor requires a sufficient number of electromagnetic fields to be created by the FG 107.

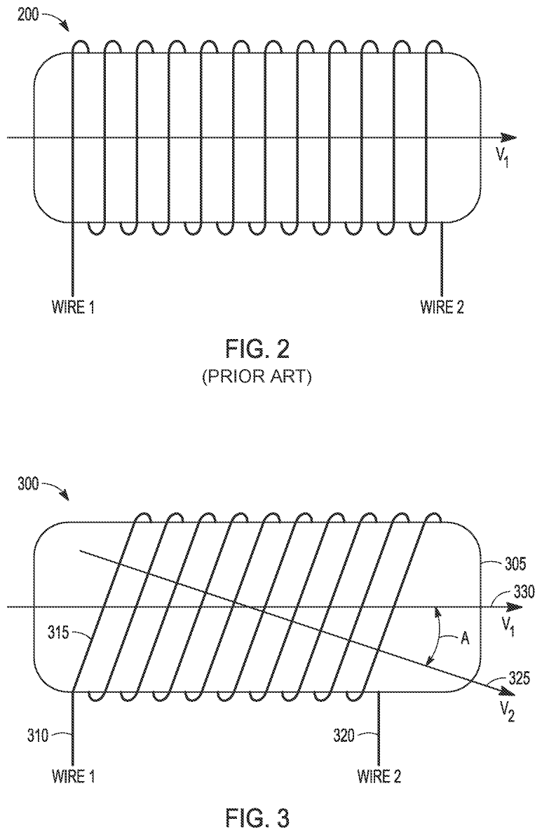

FIG. 2 is a PRIOR ART diagram of a sensor 200 having a straight wound coil. The coil sensor orientation is the same as the winding orientation. The roll degree of freedom cannot be determined from the electromagnetic field measurements from a single coil.

Sensor 200 consisting of a single coil defines only a single spatial direction, which usually coincides with the direction of the coil winding. However, in many cases the knowledge of the roll angle of the tool 112 is also required. The roll computation must include at least two vectors, which usually is done by combining two or more single coils into a sensor assembly. This combination tends to make the sensors bulky and is not suitable for sensors which are wrapped around the tools.

FIG. 3 is a diagram of a sensor 300 having a coil 315 wound at an angle “A” to a core 305 axis according to an example embodiment. The core 305 may be aligned with a tool, whose position and orientation are to be determined. In one example, a portion of the tool itself may serve as the core. A sensor winding orientation V2 is represented as a vector 325 which is at an angle to the windings direction of the coil. The sensor orientation V2 will differ from the core axis orientation V1 as represented at vector 330 which are at an angle “A” from each other. In one example A may be varied between greater than zero and less than 90 degrees, with typical ranges somewhere between 3 and 87 degrees. The coil 315 may be coupled at each end by wires 310 and 320 to provide and measure current flowing through the coil. Wires 310 and 320 may be a twisted pair of wires to provide signals to the control unit 110.

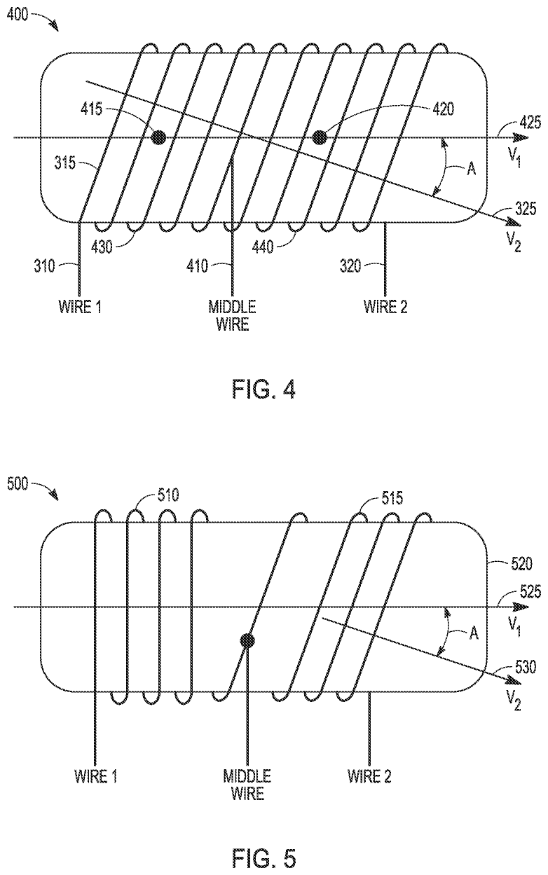

FIG. 4 is a diagram of a sensor 400 having a core orientation V1 represented as vector 425 determined as a vector connecting centers 415 and 420 of two sub-coils 430 and 440 that are defined by a middle wire 410 connected to the coil 315 at or near a middle of the coil 315. The core orientation vector 425, which is the same as the winding direction, and sensor orientation vector 325 form two vectors forming an angle “A” allowing determination of a roll degree of freedom. The angle “A” can be somewhere between 3 and 87 degrees. Dots are drawn at approximate centers 415 and 420 of each sub-coil 430 and 440.

The term middle wire is meant to signify that the wire is connected anywhere between conductive ends of the coils, and not that it is exactly half-way between the conductive ends of the coils. In further examples, a coil may be divided into more than two sub-coils by multiple middle wires.

FIG. 5 is a diagram of a sensor 500 having two sub-coils 510, 515 wound at different orientations with respect to a core 520. A first sub-coil 510 orientation is V1 525 can be the same or different as a core orientation, and a second sub-coil 515 orientation is V2 530. The different orientations of the two sub-coils 510, 515 facilitate determination of a roll degree of freedom.

As shown in FIGS. 3, 4, and 5 the sensor coil is wrapped at an angle to the core or tool axis making a slanted coil. Connecting an additional wire 410 to the middle of the coil, forms two sub-coils that can be localized separately, Localization may be used to determine positions and orientations of the sub-coils. The vector connecting the centers of the two sub-coil's positions can be used to determine the roll degree of freedom. Alternatively, if the orientations of the two sub-coils are different, they could be used to determine the roll degree of freedom.

Let the normalized vector connecting the two sub-coils centers be called V₁ and the vector of the coil sensor orientation be called V₂. Alternatively, vector V₁ can represent the orientation of the first sub-coil if it is substantially different from the orientation of the second sub-coil V₂. These two vectors define a plane. Using vectors V₁ and V₂, the third vector can be defined as their normalized vector product:

$${V}_{3} = \frac{{V}_{1} \times {V}_{2}}{\vert {V}_{1} \times {V}_{2} \vert} , \qquad ( 1 )$$

$$and$$

$${V}_{2}^{′} = \frac{{V}_{3} \times {V}_{1}}{\vert {V}_{3} \times {V}_{1} \vert} . \qquad ( 2 )$$

Vectors V₁, V₂′ and V₃ define the three orthonormal vectors which fully determine the tool orientation. These vectors can be converted into the yaw, pitch, and roll angles using standard formulas. Such standard formulas utilize well known Euler angles and may be executed via computing circuitry. Euler males can be defined by elemental geometry or by composition of rotations. The geometrical definition demonstrates that three composed elemental rotations (rotations about the axes of a coordinate system) are always sufficient to reach any target frame.

The three elemental rotations may be extrinsic (rotations about the axes xyz of the original coordinate system, which is assumed to remain motionless), or intrinsic (rotations about the axes of the rotating coordinate system XYZ, solidary with the moving body, which changes its orientation after each elemental rotation).

### EXAMPLES

Sensor coils wrapped around various cores or without cores or around tools.

Sensor coils wrapped at an angle to the core or tool axis forming a slanted coil.

An additional wire or wires connected to the middle or somewhere between the two ends of the slanted coil allowing formation of two or more sub-coils for independent localization.

A sensor coil where a full slanted coil is wound as two or more independent coils, then physically joined into one coil.

A sensor coil where the signals from the two or more sub-coils are combined in the analog circuitry or digitally to form one single stronger coil signal.

The angle of the coil can be determined by a coil localization process, which solves for the coil position and orientation.

A twisted wire pair of conductive wires may be connected to each sub-coil.

A sensor coil where a three-wire twisted triplet is connected to the sub-coils (one wire connected to one end of one sub-coil, one wire connected to the inside edge of each sub-coil, and one wire attached to the other end of the other sub-coil)

The vector or vectors connecting the two or more sub-coils' position may be determined from localizations of the sub-coils.

The vector or vectors can be used together with the coil orientation vector to determine the roll degree of freedom of the sensor consisting of the two sub-coils.

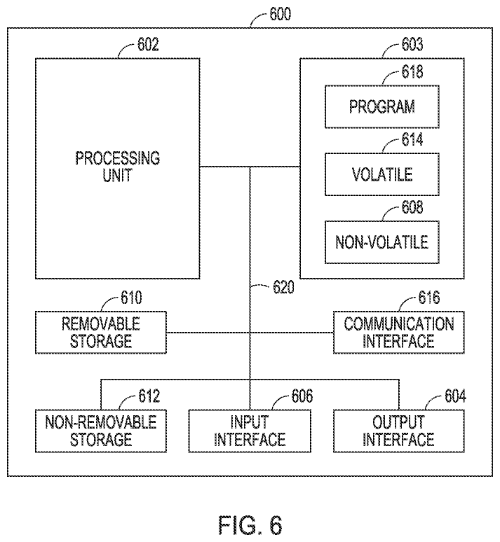

FIG. 6 is a block schematic diagram of a computer system 600 to drive antennas, collect sensor information, derive position and orientation information and for performing methods and algorithms according to example embodiments. All components need not be used in various embodiments.

One example computing device in the form of a computer 600 may include a processing unit 602, memory 603, removable storage 610, and non-removable storage 612. Although the example computing device is illustrated and described as computer 600, the computing device may be in different forms in different embodiments. For example, the computing device may instead be a smartphone, a tablet, smartwatch, smart storage device (SSD), or other computing device including the same or similar elements as illustrated and described with regard to FIG. 6. Devices, such as smartphones, tablets, and smartwatches, are generally collectively referred to as mobile devices or user equipment.

Although the various data storage elements are illustrated as part of the computer 600, the storage may also or alternatively include cloud-based storage accessible via a network, such as the Internet or server-based storage, Note also that an SSD may include a processor on which the parser may be run, allowing transfer of parsed, filtered data through I/O channels between the SSD and main memory.

Memory 603 may include volatile memory 614 and non-volatile memory 608. Computer 600 may include—or have access to a computing environment that includes—a variety of computer-readable media, such as volatile memory 614 and non-volatile memory 608, removable storage 610 and non-removable storage 612. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 600 may include or have access to a computing environment that includes input interface 606, output interface 604, and a communication interface 616. Output interface 604 may include a display device, such as a touchscreen, that also may serve as an input device. The input interface 606 may include one or more of a touchscreen, touchpad, mouse, keyboard, camera, one or more device-specific buttons, one or more sensors integrated within or coupled via wired or wireless data connections to the computer 600, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 600 are connected with a system bus 620.

Computer-readable instructions stored on a computer-readable medium are executable by the processing unit 602 of the computer 600, such as a program 618. The program 618 in some embodiments comprises software to implement one or more methods described herein, A hard drive, CD-ROM, and RAM are some examples of articles including a non-transitory computer-readable medium such as a storage device. The terms computer-readable medium, machine readable medium, and storage device do not include carrier waves or signals to the extent carrier waves and signals are deemed too transitory. Storage can also include networked storage, such as a storage area network (SAN). Computer program 618 along with the workspace manager 622 may be used to cause processing unit 602 to perform one or more methods or algorithms described herein.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. An electromagnetic field sensor comprising:
a core having a core axis forming a core vector;
a conductive coil winding supported by the core;
one or more middle conductors coupled to the conductive coil winding between a first and second end conductors of the conductive coil winding;
a first portion of the conductive coil winding having a first portion coil vector at an angle greater than zero from the core vector; and
a second portion of the conductive coil winding having a second portion coil vector at an angle equal to or greater than zero from the core vector.

2. The electromagnetic field sensor of claim 1 wherein the first and the second portion coil vectors coincide and form an angle with the core vector between 3 and 87 degrees.

3. The electromagnetic field sensor of claim 1 wherein the second portion coil vector coincides with the core vector and the first portion coil vector forms an angle with the core vector between 3 and 87 degrees.

4. The electromagnetic field sensor of claim 1 wherein the core comprises a portion of a tool.

5. The electromagnetic field sensor of claim 1 wherein the core is supported within or around a tool.

6. The electromagnetic field sensor of claim 1 and further comprising a control unit coupled to receive electrical signals from the first and second end conductors of the conductive coil winding and middle conductors to determine a position and orientation of the electromagnetic field sensor in response to actuation of a field generator.

7. The electromagnetic field sensor of claim 1 wherein the first and second portions of the coil are independently wound and joined together.

8. A sensor comprising:
a core oriented along a first vector corresponding to an axis of the core;
a coil having windings around the core at an angle to the axis of the core along a second vector;
one or more middle conductors coupled to the windings to divide the windings into two or more sub-coils about the middle conductors; and
a pair of end conductors coupled to opposite ends of the coil such that signals obtained from the middle and end conductors provide sufficient information to determine roll about the core axis.

9. The sensor of claim 8 wherein the coil comprises two or more sub-coils wound at an angle to the axis of the core to provide sufficient information to determine a relative angle between the vectors connecting the sub-coils and the sub-coil orientation vectors.

10. The sensor of claim 8 wherein the core comprises a portion of a tool or wherein the core is independent from the tool and can be placed either within or around the tool.

11. The sensor of claim 8 and further comprising a control unit coupled to receive electrical signals from the first and second end conductors of the coil winding and middle conductors to determine a position and orientation of the electromagnetic field sensor in response to actuation of a field generator.

12. The sensor of claim 11 wherein the control unit utilizes vectors V₁ and V₂, the first and second vectors, to define a vector V₃ as their normalized vector product:
$${V}_{3} = \frac{{V}_{1} \times {V}_{2}}{\vert {V}_{1} \times {V}_{2} \vert} \qquad ( 1 )$$
$$and$$
$${V}_{2}^{′} = \frac{{V}_{3} \times {V}_{1}}{\vert {V}_{3} \times {V}_{1} \vert} \qquad ( 2 )$$
vectors V₁, V₂′ and V₃ define three orthogonal vectors.

13. The sensor of claim 8 wherein the angle between the core vector and the coil vectors is between 3 and 87 degrees.

14. A sensor comprising:
a core oriented along a first vector corresponding to an axis of the core;
a coil having windings around the core at an angle to the axis of the core along a second vector;
one or more middle conductors coupled to the windings to divide the windings into two or more sub-coils about the middle conductors, wherein the sub-coils are wound at different angles from one another and either aligned with or at an angle to the axis of the core to provide sufficient information to determine a relative angle between the sub-coils; and
a pair of end conductors coupled to opposite ends of the coil such that signals obtained from the middle and end conductors provide sufficient information to determine roll about the core axis.

15. The sensor of claim 14 wherein the core comprises a portion of a tool or wherein the core is independent from the tool and can be placed either within or around the tool.

16. The sensor of claim 14 and further comprising a control unit coupled to receive electrical signals from the first and second end conductors of the coil winding and middle conductors to determine a position and orientation of the electromagnetic field sensor in response to actuation of a field generator.

17. The sensor of claim 16 wherein the control unit utilizes vectors V₁ and V₂, the first and second vectors, to define a vector V₃ as their normalized vector product:
$${V}_{3} = \frac{{V}_{1} \times {V}_{2}}{\vert {V}_{1} \times {V}_{2} \vert} \qquad ( 1 )$$
$$and$$
$${V}_{2}^{′} = \frac{{V}_{3} \times {V}_{1}}{\vert {V}_{3} \times {V}_{1} \vert} \qquad ( 2 )$$
vectors V₁, V₂′ and V₃ define three orthogonal vectors.

18. The sensor of claim 14 wherein the angle between sub coil vectors is between 3 and 87 degrees.
